(19) United States
(12) Patent Application Publication
(10) Pub. No.: US 2022/0381725 A1
(43) Pub. Date: Dec. 1, 2022

(54) CARBON DIOXIDE GAS SENSOR AND GAS SENSOR ELEMENT

(71) Applicant: ROHM CO., LTD., Kyoto (JP)

(72) Inventors: Shunsuke AKASAKA, Kyoto (JP); Ken NAKAHARA, Kyoto (JP)

(21) Appl. No.: 17/664,492

(22) Filed: May 23, 2022

(30) Foreign Application Priority Data
May 27, 2021 (JP) 2021-089145

(51) Int. Cl. G01N 27/407

(52) U.S. Cl. CPC G01N 27/4074

(57) ABSTRACT

There is provided a carbon dioxide gas sensor that includes a flow path including an inlet into which a detected target gas is introduced; and a first element and at least one second element arranged in the flow path. The first element includes a first solid electrolyte layer, a first cathode, and a first anode, the first solid electrolyte layer being interposed between the first cathode and the first anode. The at least one second element includes a second solid electrolyte layer, a second cathode, and a second anode, the second solid electrolyte layer being interposed between the second cathode and the second anode. The first solid electrolyte layer and the second solid electrolyte layer are formed of an oxygen ion conductor. The first cathode is inside the flow path. The second cathode and the second anode are inside the flow path and outside the flow path, respectively.

### CROSS-REFERENCE TO RELATED APPLICATION

This application is based upon and claims the benefit of priority from Japanese Patent Application No. 2021-089145, filed on May 27, 2021, the entire contents of which are incorporated herein by reference.

### TECHNICAL FIELD

The present disclosure relates to a carbon dioxide gas sensor and a gas sensor element.

### BACKGROUND

There has been disclosed a semiconductor carbon dioxide gas sensor in the related art.

The carbon dioxide gas sensor disclosed in the related art also responds to gases (for example, a hydrogen gas) other than a carbon dioxide gas. That is, the carbon dioxide gas sensor disclosed in the related art has low gas selectivity for the carbon dioxide gas.

### SUMMARY

The present disclosure has been made in view of the problems of the prior art as described above. More specifically, some embodiments of the present disclosure provide a carbon dioxide gas sensor with improved gas selectivity for a carbon dioxide gas.

According to one embodiment of the present disclosure, there is provided a carbon dioxide gas sensor that includes a flow path including an inlet into which a detected target gas is introduced; and a first element and at least one second element arranged in the flow path, wherein the first element includes a first solid electrolyte layer, a first cathode, and a first anode, the first solid electrolyte layer being interposed between the first cathode and the first anode, wherein the at least one second element includes a second solid electrolyte layer, a second cathode, and a second anode, the second solid electrolyte layer being interposed between the second cathode and the second anode, wherein the first solid electrolyte layer and the second solid electrolyte layer are formed of an oxygen ion conductor, wherein the first cathode is inside the flow path, and wherein the second cathode and the second anode are inside the flow path and outside the flow path, respectively.

### BRIEF DESCRIPTION OF DRAWINGS

The accompanying drawings, which are incorporated in and constitute a part of the specification, illustrate embodiments of the present disclosure.

FIG. 1 is a schematic view of a carbon dioxide gas sensor 100.

FIG. 2 is a cross-sectional view of a first element 20.

FIG. 3 is a process diagram showing a method of manufacturing the first element 20.

FIG. 4A is a cross-sectional view for explaining a first insulating layer forming step S2.

FIG. 4B is a cross-sectional view for explaining a first wiring forming step S3.

FIG. 4C is a cross-sectional view for explaining a second insulating layer forming step S4.

FIG. 4D is a cross-sectional view for explaining a second wiring forming step S5.

FIG. 4E is a cross-sectional view for explaining a third insulating layer forming step S6.

FIG. 4F is a cross-sectional view for explaining a porous oxide layer forming step S7 and a cathode forming step S8.

FIG. 4G is a cross-sectional view for explaining a solid electrolyte layer forming step S9.

FIG. 4H is a cross-sectional view for explaining a patterning step S10.

FIG. 4I is a cross-sectional view for explaining a fourth insulating layer forming step S11.

FIG. 4J is a cross-sectional view for explaining an anode forming step S12.

FIG. 5 is a cross-sectional view of a second element 30.

FIG. 6 is a process diagram showing a method of manufacturing the second element 30.

FIG. 7A is a cross-sectional view for explaining a first insulating layer forming step S15.

FIG. 7B is a cross-sectional view for explaining a wiring forming step S16.

FIG. 7C is a cross-sectional view for explaining a second insulating layer forming step S17.

FIG. 7D is a cross-sectional view for explaining a through-hole forming step S18.

FIG. 7E is a cross-sectional view for explaining a porous oxide layer forming step S19 and an anode forming step S20.

FIG. 7F is a cross-sectional view for explaining a solid electrolyte layer forming step S21.

FIG. 7G is a cross-sectional view for explaining a patterning step S22.

FIG. 7H is a cross-sectional view for explaining a third insulating layer forming step S23.

FIG. 7I is a cross-sectional view for explaining a cathode forming step S24.

FIG. 8 is a schematic view of a carbon dioxide gas sensor 100A.

FIG. 9 is an enlarged plan view of a first element 20 used in the carbon dioxide gas sensor 100A.

FIG. 10 is a cross-sectional view of the first element 20 used in the carbon dioxide gas sensor 100A.

FIG. 11 is a schematic view of a carbon dioxide gas sensor 100B.

FIG. 12 is a cross-sectional view of a first element 40.

### DETAILED DESCRIPTION

Embodiments of the present disclosure will be now described in detail with reference to the drawings. Throughout the drawings, the same or corresponding parts are denoted by the same reference numerals, and explanation thereof will not be repeated.

### First Embodiment

A carbon dioxide gas sensor according to a first embodiment (hereinafter referred to as “carbon dioxide gas sensor 100”) will be described.

<Configuration of Carbon Dioxide Gas Sensor 100>

The configuration of the carbon dioxide gas sensor 100 will be described below.

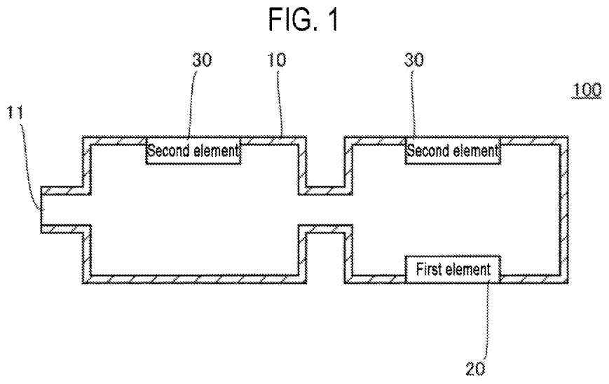

FIG. 1 is a schematic view of the carbon dioxide gas sensor 100. As shown in FIG. 1, the carbon dioxide gas sensor 100 includes a flow path 10, a first element 20, and a plurality of second elements 30. In an example shown in FIG. 1, the carbon dioxide gas sensor 100 includes two second elements 30. However, the number of second elements 30 may be one.

The flow path 10 includes an inlet 11. A gas to be detected (detection target gas) is introduced from the inlet 11. The flow path 10 includes two internal spaces. The detection target gas includes a carbon dioxide gas, water vapor, an oxygen gas, and a nitrogen oxide gas.

The internal space of the flow path 10 near the inlet 11 is referred to as a first internal space, and the internal space of the flow path 10 farther from the inlet 11 than the first internal space is referred to as a second internal space. The first element 20 is arranged in the second internal space. One of the plurality of second elements 30 is arranged in the first internal space. From another point of view, one of the plurality of second elements 30 is arranged on the upstream side of the first element 20 in the flow path 10.

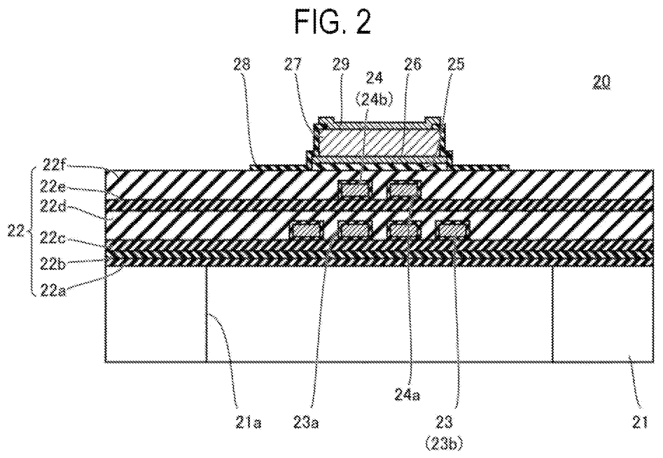

FIG. 2 is a cross-sectional view of the first element 20. As shown in FIG. 2, the first element 20 includes a substrate 21, an insulating layer 22, a wiring 23, a wiring 24, a porous oxide layer 25, a cathode 26, a solid electrolyte layer 27, an insulating layer 28, and an anode 29.

The substrate 21 is formed of, for example, single crystal silicon. A cavity 21a is formed in the substrate 21. The cavity 21a passes through the substrate 21 along a thickness direction thereof. The insulating layer 22 is arranged on the substrate 21. A portion of the insulating layer 22 on the cavity 21a may be referred to as a membrane portion of the insulating layer 22.

The insulating layer 22 includes a first layer 22a, a second layer 22b, a third layer 22c, and a fourth layer 22d. The first layer 22a, the third layer 22c, and the fourth layer 22d are formed of, for example, silicon oxide. The second layer 22b is formed of, for example, silicon nitride. The first layer 22a is arranged on the substrate 21. The second layer 22b is arranged on the first layer 22a. The third layer 22c is arranged on the second layer 22b. The fourth layer 22d is arranged on the third layer 22c.

The insulating layer 22 further includes a fifth layer 22e and a sixth layer 22f. The fifth layer 22e is formed of silicon nitride, and the sixth layer 22f is formed of silicon oxide. The fifth layer 22e is arranged on the fourth layer 22d, and the sixth layer 22f is arranged on the fifth layer 22e.

The wiring 23 is formed of, for example, platinum. The wiring 23 is arranged in the insulating layer 22. More specifically, the wiring 23 is arranged on the third layer 22c and is covered with the fourth layer 22d. The periphery of the wiring 23 is covered with an adhesion layer 23a. The adhesion layer 23a is formed of, for example, titanium oxide. The adhesion between the wiring 23 and the insulating layer 22 (the third layer 22c and the fourth layer 22d) is secured by the adhesion layer 23a.

The wiring 23 includes a heater part 23b. The heater part 23b is arranged in the membrane portion of the insulating layer 22. A portion of the wiring 23 constituting the heater part 23b meanders in a plan view. When a current flows through the wiring 23, the heater part 23b generates heat due to resistance, so that the solid electrolyte layer 27 is heated.

The wiring 24 is formed of, for example, platinum. The wiring 24 is arranged in the insulating layer 22. More specifically, the wiring 24 is arranged on the fifth layer 22e and is covered with the sixth layer 22f. The periphery of the wiring 24 is covered with an adhesion layer 24a. The adhesion layer 24a is formed of, for example, titanium oxide. The adhesion between the wiring 24 and the insulating layer 22 (the fifth layer 22e and the sixth layer 220 is secured by the adhesion layer 24a.

The wiring 24 includes a temperature sensor part 24b. A portion of the wiring 24 constituting the temperature sensor part 24b meanders in a plan view. The temperature sensor part 24b is located at a position overlapping the heater part 23b in a plan view. The temperature sensor part 24b functions as a resistance thermometer bulb. That is, a temperature in the vicinity of the temperature sensor part 24b is measured by measuring a change in an electric resistance value of the wiring 24 constituting the temperature sensor part 24b.

The porous oxide layer 25 is arranged on the membrane portion of the insulating layer 22. The porous oxide layer 25 is formed of, for example, tantalum oxide. Since the porous oxide layer 25 is porous, it serves as a flow path through which the detection target gas flows.

The cathode 26 is a layer formed of porous metal. The cathode 26 is formed of, for example, platinum. The cathode 26 is arranged on the porous oxide layer 25. The solid electrolyte layer 27 is formed of an oxygen ion conductor. A specific example of the oxygen ion conductor may include yttria-stabilized zirconia. The yttria-stabilized zirconia exhibits oxygen ion conductivity when it is heated to about 500 degrees C. The solid electrolyte layer 27 is arranged on the cathode 26.

The insulating layer 28 is, for example, a layer in which a silicon oxide layer and a tantalum oxide layer are stacked. The insulating layer 28 is arranged on the insulating layer 22 so as to cover the porous oxide layer 25, the cathode 26, and the solid electrolyte layer 27. The insulating layer 28 is formed with an opening that exposes the solid electrolyte layer 27.

The anode 29 is arranged on a portion of the solid electrolyte layer 27, which is exposed from the opening of the insulating layer 28. The anode 29 is a layer formed of porous metal. The anode 29 is formed of, for example, platinum.

When the carbon dioxide gas in the detection target gas reaches the cathode 26 through the porous oxide layer 25, it becomes oxygen ions by receiving electrons from the cathode 26. These oxygen ions move to the anode 29 through the solid electrolyte layer 27 by a voltage between the cathode 26 and the anode 29. The oxygen ions that have moved to the anode 29 become an oxygen gas by emitting electrons to the anode 29.

A current flowing between the cathode 26 and the anode 29 due to this reaction is proportional to the concentration of the carbon dioxide gas in the detection target gas. Therefore, by detecting the current flowing between the cathode 26 and the anode 29, the first element 20 can detect the carbon dioxide concentration in the detection target gas.

The minimum value of the voltage between the cathode 26 and the anode 29 where the above reaction occurs is defined as a first voltage. A voltage equal to or higher than the first voltage is applied between the cathode 26 and the anode 29. When the cathode 26 and the anode 29 are formed of platinum, the first voltage is about 2.2 volts.

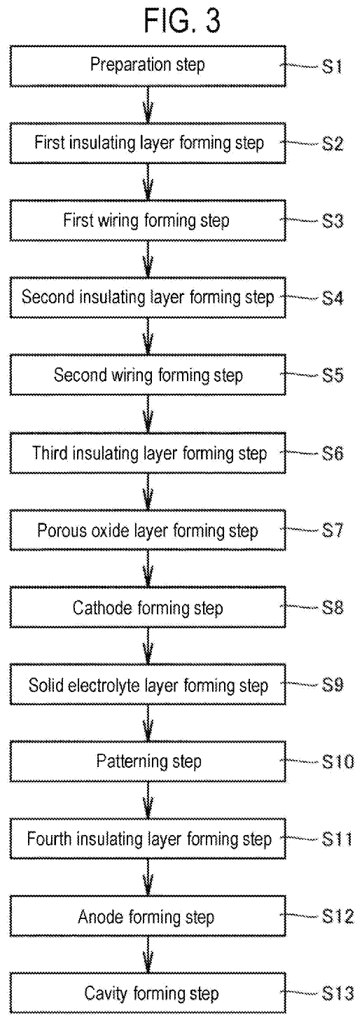

FIG. 3 is a process diagram showing a method of manufacturing the first element 20. As shown in FIG. 3, the first element 20 is formed by a preparation step S1, a first insulating layer forming step S2, a first wiring forming step S3, a second insulating layer forming step S4, a second wiring forming step S5, a third insulating layer forming step S6, a porous oxide layer forming step S7, a cathode forming step S8, a solid electrolyte layer forming step S9, a patterning step S10, a fourth insulating layer forming step S11, an anode forming step S12, and a cavity forming step S13.

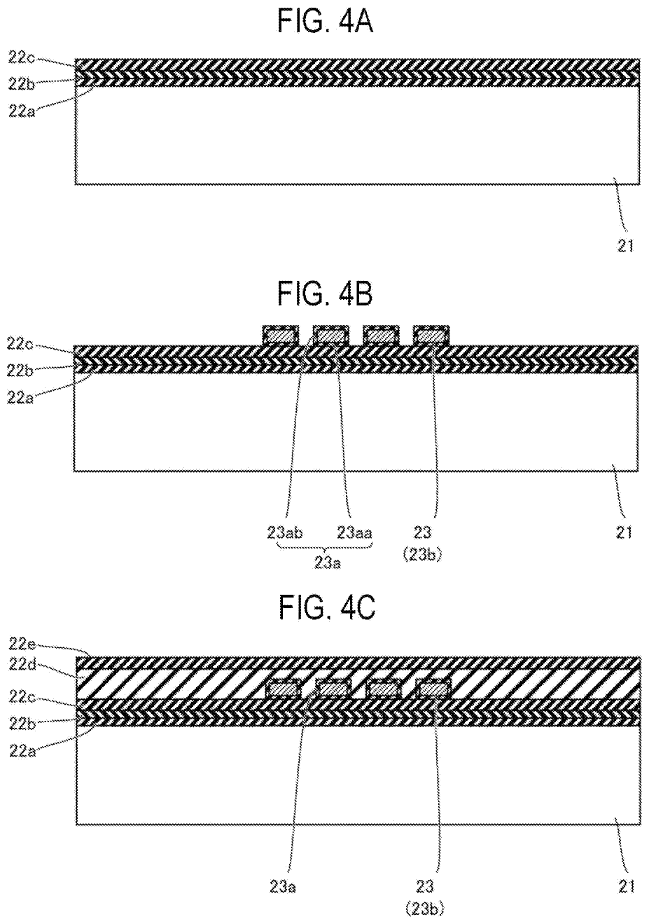

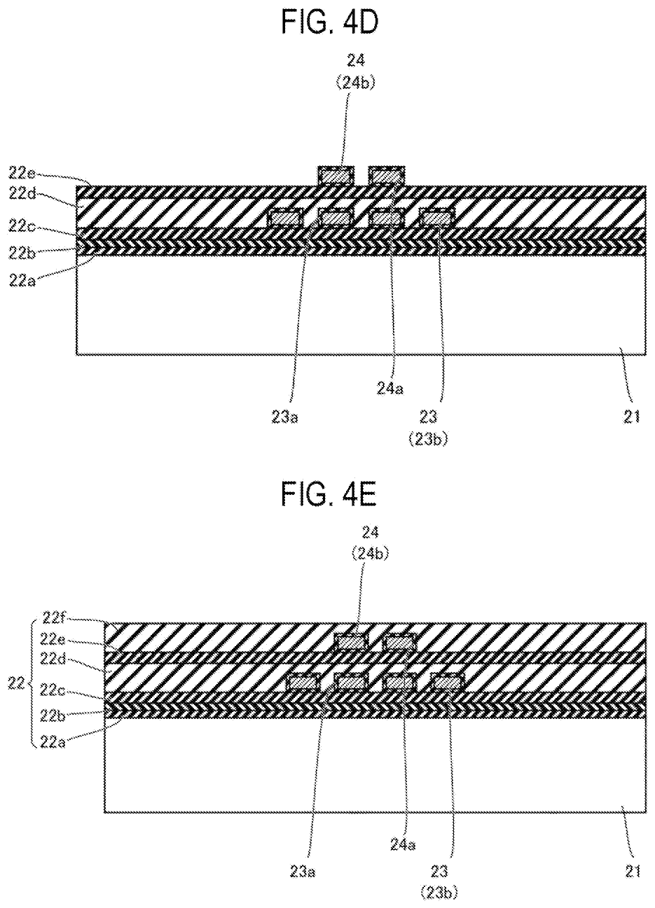

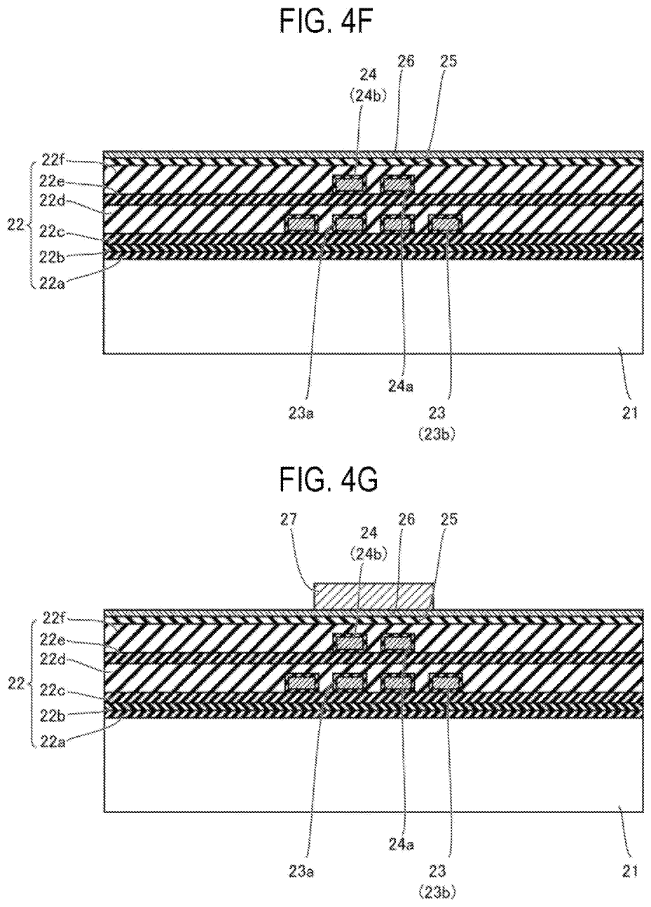

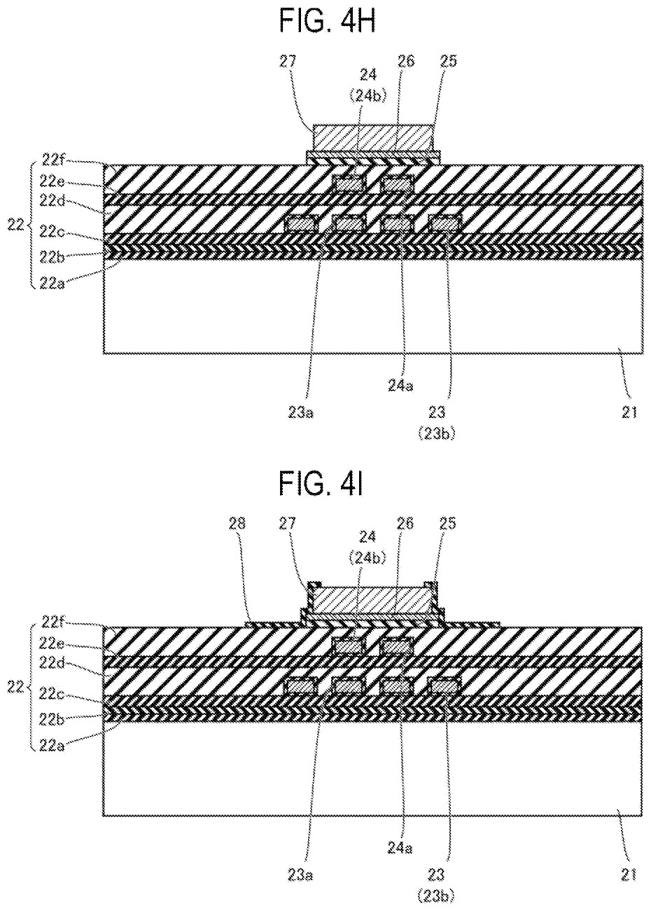

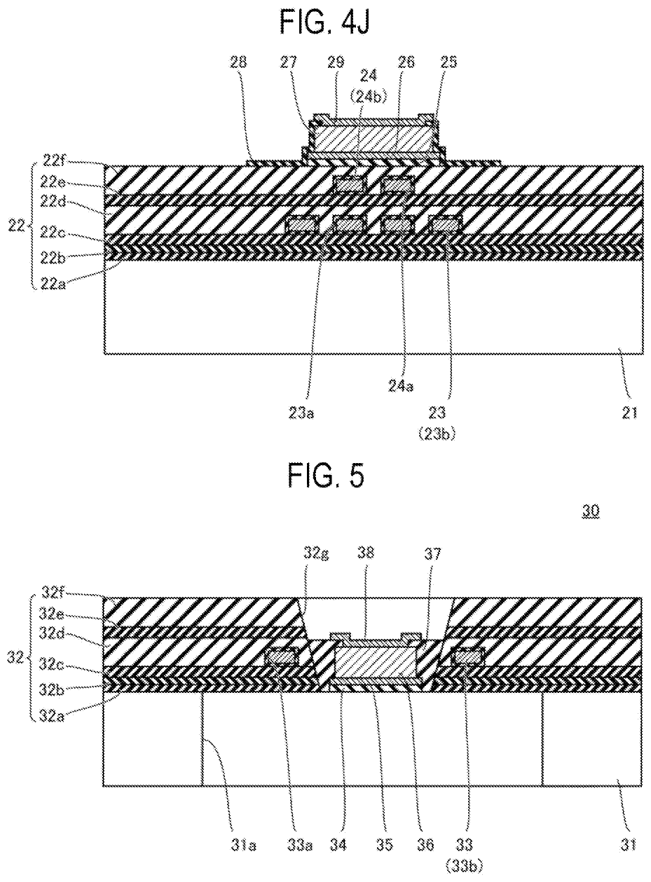

In the preparation step S1, the substrate 21 is prepared. The cavity 21a is not formed in the substrate 21 prepared in the preparation step S1. FIG. 4A is a cross-sectional view for explaining the first insulating layer forming step S2. As shown in FIG. 4A, in the first insulating layer forming step S2, the first layer 22a, the second layer 22b, and the third layer 22c are sequentially formed. The formation of the first layer 22a, the second layer 22b, and the third layer 22c is performed, for example, by using a CVD (Chemical Vapor Deposition) method.

FIG. 4B is a cross-sectional view for explaining the first wiring forming step S3. As shown in FIG. 4B, in the first wiring forming step S3, the wiring 23 and the adhesion layer 23a are formed. A portion of the adhesion layer 23a on the third layer 22c is referred to as a first portion 23aa, and a portion of the adhesion layer 23a covering the wiring 23 is referred to as a second portion 23ab. In the first wiring forming step S3, first, the first portion 23aa is formed. The first portion 23aa is formed by film-forming and patterning the constituent material of the adhesion layer 23a. This film formation is performed by, for example, sputtering. This patterning is performed by forming a mask using photolithography and etching using the mask.

In the first wiring forming step S3, second, the wiring 23 is formed. The wiring 23 is formed by forming a film with the constituent material of the wiring 23 and patterning the film. This film formation is performed by, for example, sputtering. This patterning is performed by forming a mask using photolithography and etching using the mask.

In the first wiring forming step S3, third, the second portion 23ab is formed. The second portion 23ab is formed by forming a film with the constituent material of the second portion 23ab and patterning the film. This film formation is performed by, for example, sputtering. This patterning is performed by forming a mask using photolithography and etching using the mask.

FIG. 4C is a cross-sectional view for explaining the second insulating layer forming step S4. As shown in FIG. 4C, in the second insulating layer forming step S4, for example, a CVD method is used to form the fourth layer 22d. FIG. 4D is a cross-sectional view for explaining the second wiring forming step S5. As shown in FIG. 4D, in the second wiring forming step S5, the wiring 24 is formed. A method of forming the wiring 24 is the same as the method of forming the wiring 23. FIG. 4E is a cross-sectional view for explaining the third insulating layer forming step S6. As shown in FIG. 4E, in the third insulating layer forming step S6, the fifth layer 22e and the sixth layer 22f are sequentially formed by using, for example, a CVD method.

FIG. 4F is a cross-sectional view for explaining the porous oxide layer forming step S7 and the cathode forming step S8. As shown in FIG. 4F, the porous oxide layer 25 is formed in the porous oxide layer forming step S7, and the cathode 26 is formed in the cathode forming step S8. The porous oxide layer 25 and the cathode 26 are formed by, for example, sputtering.

FIG. 4G is a cross-sectional view for explaining the solid electrolyte layer forming step S9. As shown in FIG. 4G, in the solid electrolyte layer forming step S9, the solid electrolyte layer 27 is formed. The solid electrolyte layer 27 is formed by forming a film with the constituent material of the solid electrolyte layer 27 and patterning the film. This film formation is performed by, for example, sputtering. This patterning is performed by, for example, dry etching. FIG. 4H is a cross-sectional view for explaining the patterning step S10. As shown in FIG. 4H, in the patterning step S10, the porous oxide layer 25 and the cathode 26 are patterned. This patterning is performed by, for example, dry etching.

FIG. 4I is a cross-sectional view for explaining the fourth insulating layer forming step S11. As shown in FIG. 4I, in the fourth insulating layer forming step S11, the insulating layer 28 is formed. The formation of the insulating layer 28 is performed by, for example, sputtering. The insulating layer 28 is formed with an opening for partially exposing the solid electrolyte layer 27 by etching. FIG. 4J is a cross-sectional view for explaining the anode forming step S12. As shown in FIG. 4J, in the anode forming step S12, the anode 29 is formed. The anode 29 is formed by forming a film with the constituent material of the anode 29. This film formation is performed by, for example, sputtering. This patterning is performed by, for example, dry etching.

In the cavity forming step S13, the cavity 21a is formed by, for example, wet etching. From the above, the first element 20 having the structure shown in FIG. 2 is formed.

FIG. 5 is a cross-sectional view of the second element 30. As shown in FIG. 5, the second element 30 includes a substrate 31, an insulating layer 32, a wiring 33, a porous oxide layer 34, an anode 35, a solid electrolyte layer 36, an insulating layer 37, and a cathode 38.

The substrate 31 is formed of, for example, single crystal silicon. A cavity 31a is formed in the substrate 31. The cavity 31a passes through the substrate 31 along the thickness direction. The insulating layer 32 is arranged on the substrate 31. A portion of the insulating layer 32 on the cavity 31a may be referred to as a membrane portion of the insulating layer 32.

The insulating layer 32 includes a first layer 32a, a second layer 32b, a third layer 32c, and a fourth layer 32d. The first layer 32a, the third layer 32c, and the fourth layer 32d are formed of, for example, silicon oxide. The second layer 32b is formed of, for example, silicon nitride. The first layer 32a is arranged on the substrate 31. The second layer 32b is arranged on the first layer 32a. The third layer 32c is arranged on the second layer 32b. The fourth layer 32d is arranged on the third layer 32c.

The insulating layer 32 further includes a fifth layer 32e and a sixth layer 32f. The fifth layer 32e is formed of silicon nitride, and the sixth layer 32f is formed of silicon oxide. The fifth layer 32e is arranged on the fourth layer 32d, and the sixth layer 32f is arranged on the fifth layer 32e. A through-hole 32g is formed in the membrane portion of the insulating layer 32. The through-hole 32g is configured to communicate with the cavity 31a. The through-hole 32g is formed in a tapered shape whose inner diameter decreases, for example, as it approaches the cavity 31a side.

The wiring 33 is formed of, for example, platinum. The wiring 33 is arranged in the insulating layer 32. More specifically, the wiring 33 is arranged on the third layer 32c and is covered with the fourth layer 32d. The periphery of the wiring 33 is covered with an adhesion layer 33a. The adhesion layer 33a is formed of, for example, titanium oxide. As a result, the adhesion between the wiring 33 and the insulating layer 32 (the third layer 32c and the fourth layer 32d) is secured by the adhesion layer 33a.

The wiring 33 includes a heater part 33b. The heater part 33b is arranged in the membrane portion of the insulating layer 32. A portion of the wiring 33 constituting the heater part 33b is arranged around the through-hole 32g.

The porous oxide layer 34 is arranged on the membrane portion of the insulating layer 32. The porous oxide layer 34 is formed of, for example, tantalum oxide. The anode 35 is a layer formed of porous metal. The anode 35 is formed of, for example, platinum. The anode 35 is arranged on the porous oxide layer 34. The solid electrolyte layer 36 is formed of an oxygen ion conductor. A specific example of the oxygen ion conductor may include yttria-stabilized zirconia. The solid electrolyte layer 36 is arranged on the anode 35.

The insulating layer 37 is, for example, a layer in which a silicon oxide layer and a tantalum oxide layer are stacked. The insulating layer 37 covers the porous oxide layer 34, the anode 35, and the solid electrolyte layer 36. The insulating layer 37 is formed with an opening that exposes the solid electrolyte layer 36. The cathode 38 is arranged on a portion of the solid electrolyte layer 36, which is exposed from the opening of the insulating layer 37. The cathode 38 is a layer formed of porous metal. The cathode 38 is formed of, for example, platinum.

The porous oxide layer 34, the anode 35, the solid electrolyte layer 36, the insulating layer 37, and the cathode 38 are arranged in the through-hole 32g. The porous oxide layer 34 is exposed to the cavity 31a.

Water vapor, an oxygen gas, and a nitrogen oxide gas in the detection target gas become oxygen ions by receiving electrons from the cathode 38. These oxygen ions move to the anode 35 through the solid electrolyte layer 36 by a voltage between the cathode 38 and the anode 35. The oxygen ions that have moved to the anode 35 become an oxygen gas by emitting electrons to the anode 35.

When the oxygen ions are generated from the water vapor in the detection target gas, a hydrogen gas is generated at the cathode 38. Further, when the oxygen ions are generated from the nitrogen oxide in the detection target gas, a nitrogen gas is generated at the cathode 38.

The minimum value of the voltage between the cathode 38 and the anode 35 where the above reaction occurs is defined as a second voltage. A voltage equal to or higher than the second voltage is applied between the cathode 38 and the anode 35. However, since the cathode 38 does not generate oxygen ions from a carbon dioxide gas in the detection target gas, the voltage between the cathode 38 and the anode 35 is lower than the first voltage. When the cathode 38 and the anode 35 are formed of platinum, the second voltage is about 1.2 volts.

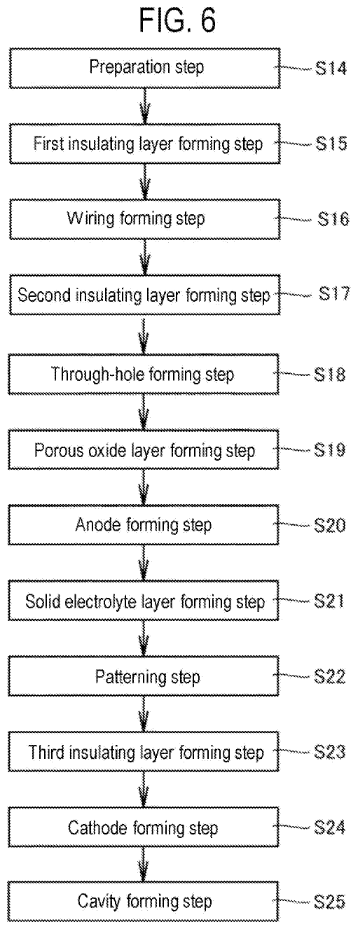

FIG. 6 is a process diagram showing a method of manufacturing the second element 30. As shown in FIG. 6, the second element 30 is formed by a preparation step S14, a first insulating layer forming step S15, a wiring forming step S16, a second insulating layer forming step S17, a through-hole forming step S18, a porous oxide layer forming step S19, an anode forming step S20, a solid electrolyte layer forming step S21, a patterning step S22, a third insulating layer forming step S23, a cathode forming step S24, and a cavity forming step S25.

In the preparation step S14, the substrate 31 is prepared. The cavity 31a is not formed in the substrate 31 prepared in the preparation step S14.

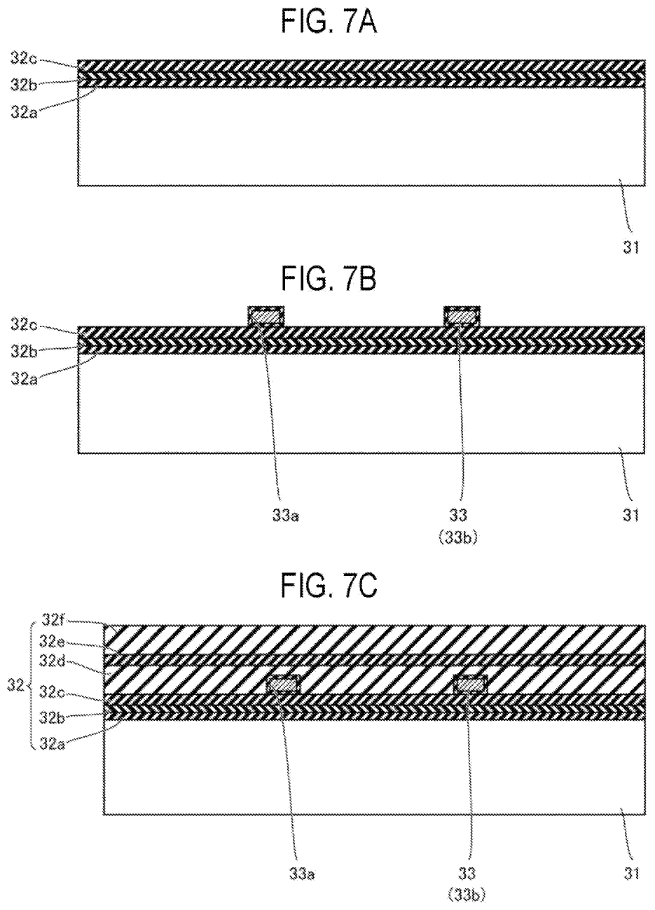

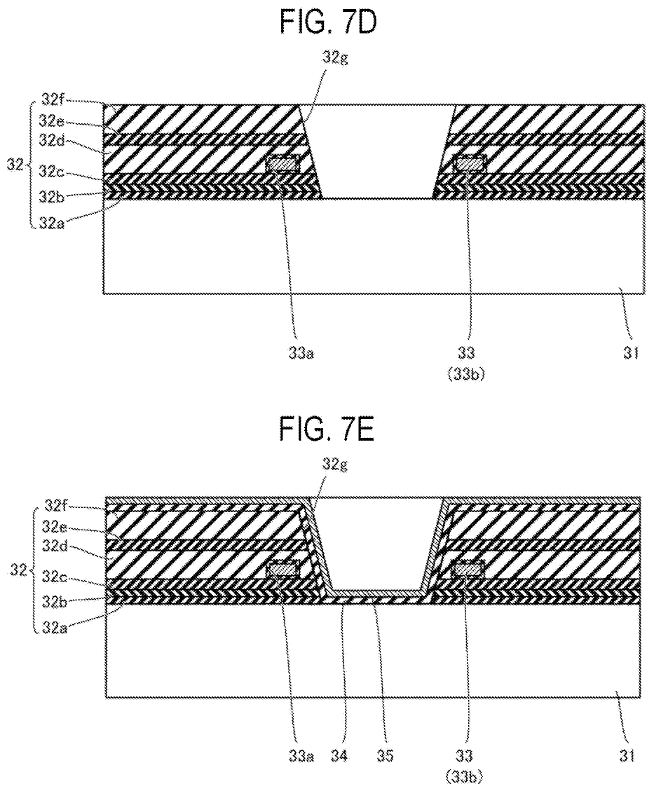

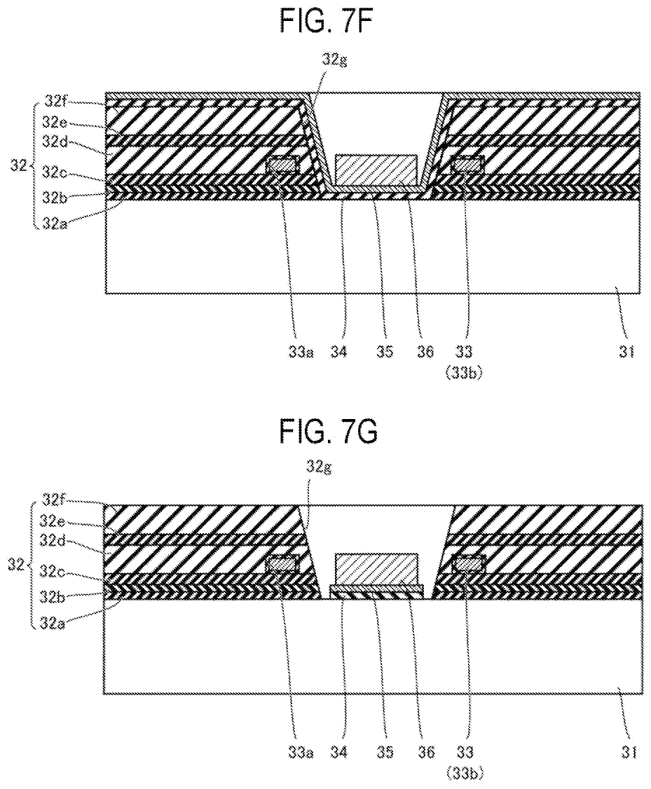

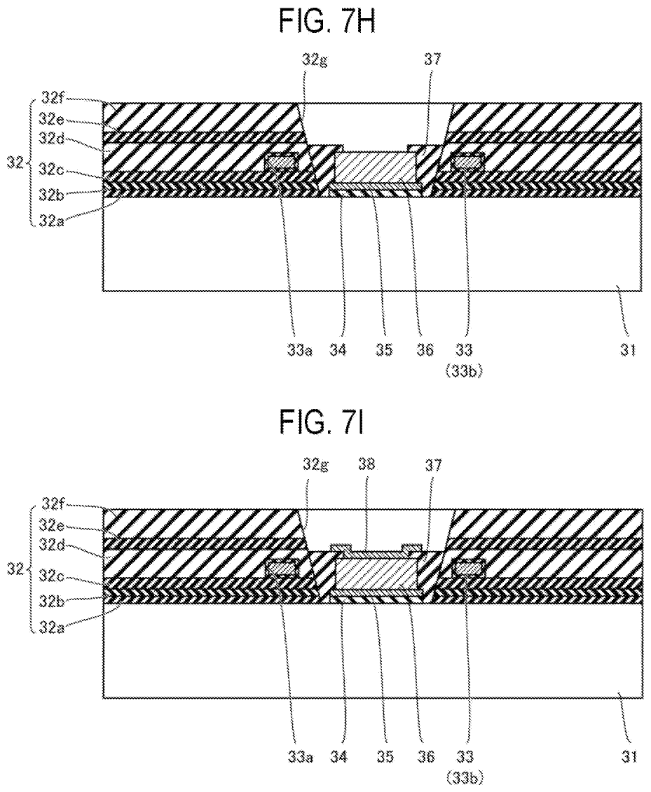

FIG. 7A is a cross-sectional view for explaining the first insulating layer forming step S15. As shown in FIG. 7A, in the first insulating layer forming step S2, the first layer 32a, the second layer 32b, and the third layer 32c are sequentially formed. A method of forming the first layer 32a, the second layer 32b, and the third layer 32c is the same as the method of forming the first layer 22a, the second layer 22b, and the third layer 22c.

FIG. 7B is a cross-sectional view for explaining the wiring forming step S16. As shown in FIG. 7B, in the wiring forming step S16, the wiring 33 and the adhesion layer 33a are formed. A method of forming the wiring 33 and the adhesion layer 33a is the same as the method of forming the wiring 23 and the adhesion layer 23a.

FIG. 7C is a cross-sectional view for explaining the second insulating layer forming step S17. As shown in FIG. 7C, in the second insulating layer forming step S17, the fourth layer 32d, the fifth layer 32e, and the sixth layer 32f are formed. A method of forming the fourth layer 32d, the fifth layer 32e, and the sixth layer 32f is the same as the method of forming the fourth layer 22d, the fifth layer 22e, and the sixth layer 22f.

FIG. 7D is a cross-sectional view for explaining the through-hole forming step S18. As shown in FIG. 7D, in the through-hole forming step S18, the through-hole 32g is formed. The formation of the through-hole 32g is performed by, for example, dry etching. FIG. 7E is a cross-sectional view for explaining the porous oxide layer forming step S19 and the anode forming step S20. As shown in FIG. 7E, the porous oxide layer 34 is formed in the porous oxide layer forming step S19, and the anode 35 is formed in the anode forming step S20. A method of forming the porous oxide layer 34 and the anode 35 is the same as the method of forming the porous oxide layer 25 and the cathode 26.

FIG. 7F is a cross-sectional view for explaining the solid electrolyte layer forming step S21. As shown in FIG. 7F, in the solid electrolyte layer forming step S21, the solid electrolyte layer 36 is formed. A method of forming the solid electrolyte layer 36 is the same as the method of forming the solid electrolyte layer 27. FIG. 7G is a cross-sectional view for explaining the patterning step S22. As shown in FIG. 7G, in the patterning step S22, the porous oxide layer 34 and the anode 35 are patterned. The patterning step S22 is performed by the same method as the patterning step S10.

FIG. 7H is a cross-sectional view for explaining the third insulating layer forming step S23. As shown in FIG. 7H, in the third insulating layer forming step S23, the insulating layer 37 is formed. The method of forming the insulating layer 37 is the same as the method of forming the insulating layer 28. The insulating layer 37 is formed with an opening that partially exposes the solid electrolyte layer 36 by etching. FIG. 7I is a cross-sectional view for explaining the cathode forming step S24. As shown in FIG. 7I, in the cathode forming step S24, the cathode 38 is formed. A method of forming the cathode 38 is the same as the method of forming the anode 29.

In the cavity forming step S25, the cavity 31a is formed. A method of forming the cavity 31a is the same as the method of forming the cavity 21a. From the above, the second element 30 having the structure shown in FIG. 5 is formed.

The first element 20 is arranged such that the cathode 26 is inside the flow path 10. The anode 29 may be located inside the flow path 10 or outside the flow path 10. The second element 30 is arranged such that the cathode 38 is inside the flow path 10 and the anode 35 is outside the flow path 10.

<Effect of Carbon Dioxide Gas Sensor 100>

The effects of the carbon dioxide gas sensor 100 will be described below.

As described above, the second element 30 is arranged such that the cathode 38 is inside the flow path 10 and the anode 35 is outside the flow path 10. The water vapor, the oxygen gas, and the nitrogen oxide gas in the detection target gas are decomposed in the cathode 38. Further, since the anode 35 is arranged outside the flow path 10, the oxygen gas generated in the anode 35 does not increase the concentration of oxygen gas in the detection target gas reaching the first element 20.

Therefore, the concentrations of water vapor, oxygen gas, and nitrogen oxide gas in the detection target gas that have reached the first element 20 are lower than those when they are introduced from the inlet 11. In this way, the first element 20 is less susceptible to the influence of water vapor, oxygen gas, and nitrogen oxide in the detection target gas when detecting the concentration of the carbon dioxide gas in the detection target gas. Therefore, the gas selectivity of the carbon dioxide gas sensor 100 for the carbon dioxide gas is improved.

When the carbon dioxide gas sensor 100 includes a plurality of second elements 30, it is possible to further reduce the concentrations of water vapor, oxygen gas, and nitrogen oxide gas in the detection target gas that has reached the first element 20. Therefore, the gas selectivity of the carbon dioxide gas sensor 100 for carbon dioxide gas is further improved.

<Modification>

When the voltage equal to or higher than the first voltage is applied between the cathode 26 and the anode 29, the example in which the first element 20 detects the concentration of the carbon dioxide gas in the detection target gas by detecting the current flowing between the cathode 26 and the anode 29 has been described in the above.

A difference between a current (first current) flowing between the cathode 26 and the anode 29 when the voltage equal to or higher than the second voltage and lower than the first voltage is applied between the cathode 26 and the anode 29 and a current (second current) flowing between the cathode 26 and the anode 29 when the voltage equal to or higher than the first voltage is applied between the cathode 26 and the anode 29 is proportional to the concentration of the carbon dioxide gas in the detection target gas. Therefore, the first element 20 may detect the concentration of the carbon dioxide gas in the detection target gas based on the difference between the first current and the second current. Further, in order to improve the sensitivity, the concentration of the carbon dioxide gas in the detection target gas may be detected by integrating the difference between the first current and the second current.

### Second Embodiment

A carbon dioxide gas sensor according to a second embodiment (hereinafter referred to as a “carbon dioxide gas sensor 100A”) will be described. Here, the differences from the carbon dioxide gas sensor 100 will be mainly described, and a duplicate explanation will not be repeated.

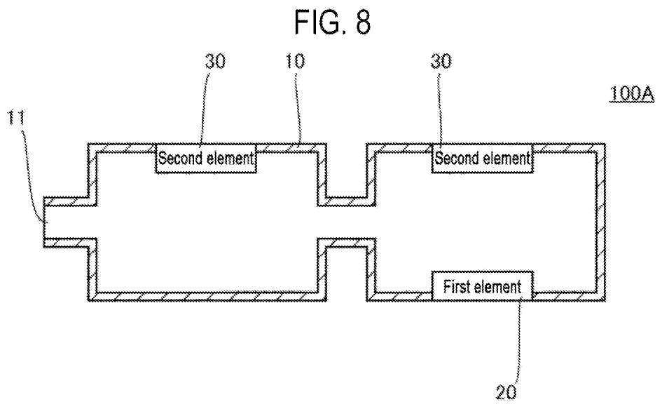

FIG. 8 is a schematic diagram of the carbon dioxide gas sensor 100A. As shown in FIG. 8, the carbon dioxide gas sensor 100A includes a flow path 10, a first element 20, and a plurality of second elements 30. In this regard, the configuration of the carbon dioxide gas sensor 100A is the same as the configuration of the carbon dioxide gas sensor 100. In the carbon dioxide gas sensor 100A, the details of the first element 20 are different from those of the carbon dioxide gas sensor 100. In this regard, the configuration of the carbon dioxide gas sensor 100A is different from the configuration of the carbon dioxide gas sensor 100.

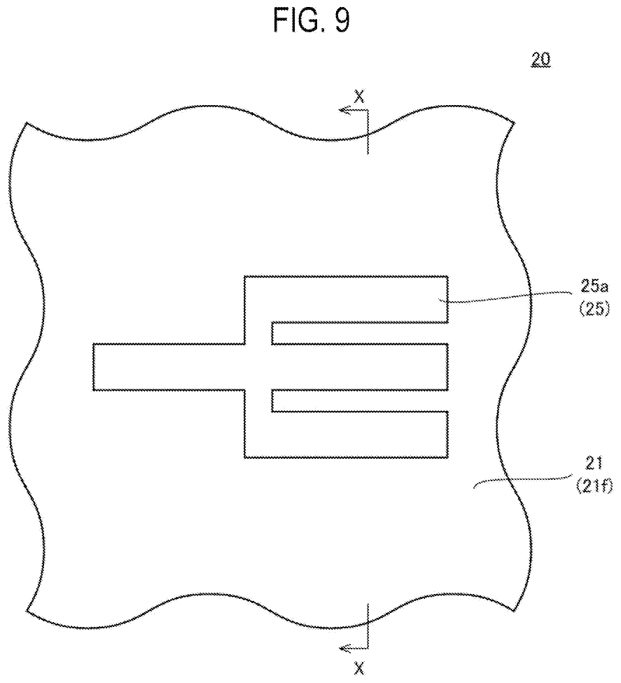

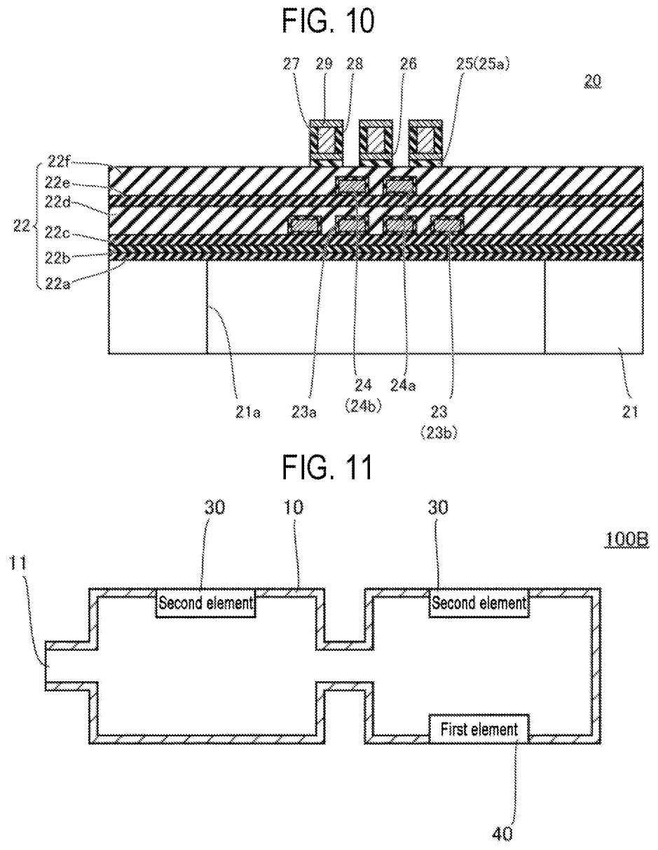

FIG. 9 is an enlarged plan view of the first element 20 used in the carbon dioxide gas sensor 100A. The cathode 26, the solid electrolyte layer 27, the insulating layer 28, and the anode 29 are not shown in FIG. 9. FIG. 10 is a cross-sectional view of the first element 20 used in the carbon dioxide gas sensor 100A. As shown in FIGS. 9 and 10, in the first element 20 used in the carbon dioxide gas sensor 100A, the porous oxide layer 25 includes a comb tooth portion 25a. The comb tooth portion 25a includes a comb tooth shape in a plan view. The insulating layer 28 is removed from the side surface of the comb tooth portion 25a.

In the first element 20 used in the carbon dioxide gas sensor 100, the side surface of the porous oxide layer 25 is covered with the insulating layer 28. Therefore, in the first element 20 used in the carbon dioxide gas sensor 100, the flow rate of the detection target gas that reaches the cathode 26 through the porous oxide layer 25 is limited, which is advantageous when the concentration of the carbon dioxide gas in the detection target gas is high.

On the other hand, in the first element 20 used in the carbon dioxide gas sensor 100A, the insulating layer 28 is removed from the side surface of the comb tooth portion 25a. Therefore, in the first element 20 used in the carbon dioxide gas sensor 100A, it is difficult to limit the flow rate of the detection target gas that reaches the cathode 26 through the porous oxide layer 25, which is advantageous for improving the sensitivity when the concentration of the carbon dioxide gas in the detection target gas is low.

### Third Embodiment

A carbon dioxide gas sensor according to a third embodiment (hereinafter referred to as a “carbon dioxide gas sensor 100B”) will be described. Here, the differences from the carbon dioxide gas sensor 100 will be mainly described, and a duplicate explanation will not be repeated.

FIG. 11 is a schematic diagram of the carbon dioxide gas sensor 100B. As shown in FIG. 11, the carbon dioxide gas sensor 100B includes a flow path 10 and a plurality of second elements 30. In this regard, the configuration of the carbon dioxide gas sensor 100B is the same as the configuration of the carbon dioxide gas sensor 100.

In the carbon dioxide gas sensor 100B, a first element 40 is used instead of the first element 20. In this regard, the configuration of the carbon dioxide gas sensor 100B is different from the configuration of the carbon dioxide gas sensor 100.

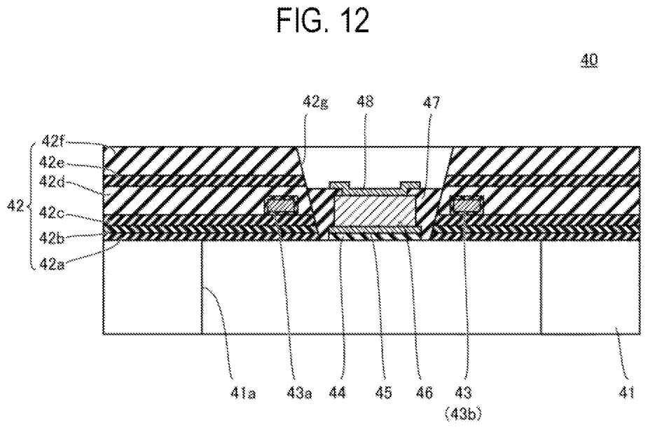

FIG. 12 is a cross-sectional view of the first element 40. As shown in FIG. 12, the first element 40 includes a substrate 41, an insulating layer 42, a wiring 43, a porous oxide layer 44, an anode 45, a solid electrolyte layer 46, an insulating layer 47, and a cathode 48.

The substrate 41 is formed of, for example, single crystal silicon. A cavity 41a is formed in the substrate 41. The cavity 41a passes through the substrate 41 along the thickness direction. The insulating layer 42 is arranged on the substrate 41. A portion of the insulating layer 42 on the cavity 41a may be referred to as a membrane portion of the insulating layer 42.

The insulating layer 42 includes a first layer 42a, a second layer 42b, a third layer 42c, and a fourth layer 42d. The first layer 42a, the third layer 42c, and the fourth layer 42d are formed of, for example, silicon oxide. The second layer 42b is formed of, for example, silicon nitride. The first layer 42a is arranged on the substrate 41. The second layer 42b is arranged on the first layer 42a. The third layer 42c is arranged on the second layer 42b. The fourth layer 42d is arranged on the third layer 42c.

The insulating layer 42 further includes a fifth layer 42e and a sixth layer 42f. The fifth layer 42e is formed of silicon nitride, and the sixth layer 42f is formed of silicon oxide. The fifth layer 42e is arranged on the fourth layer 42d, and the sixth layer 42f is arranged on the fifth layer 42e. A through-hole 42g is formed in the membrane portion of the insulating layer 42. The through-hole 42g is configured to communicate with the cavity 41a. The through-hole 42g is formed in a tapered shape whose inner diameter decreases, for example, as it approaches the cavity 41a side.

The wiring 43 is formed of, for example, platinum. The wiring 43 is arranged in the insulating layer 42. More specifically, the wiring 43 is arranged on the third layer 42c and is covered with the fourth layer 42d. The periphery of the wiring 43 is covered with an adhesion layer 43a. The adhesion layer 43a is formed of, for example, titanium oxide. As a result, the adhesion between the wiring 43 and the insulating layer 42 (the third layer 42c and the fourth layer 42d) is secured by the adhesion layer 43a.

The wiring 43 has a heater part 43b. The heater part 43b is arranged in the membrane portion of the insulating layer 42. A portion of the wiring 43 constituting the heater part 43b is arranged around the through-hole 42g.

The porous oxide layer 44 is arranged on the membrane portion of the insulating layer 42. The porous oxide layer 44 is formed of, for example, tantalum oxide. The anode 45 is a layer formed of porous metal. The anode 45 is formed of, for example, platinum. The anode 45 is arranged on the porous oxide layer 44. The solid electrolyte layer 46 is formed of an oxygen ion conductor. A specific example of the oxygen ion conductor may include yttria-stabilized zirconia. The solid electrolyte layer 46 is arranged on the anode 45.

The insulating layer 47 is, for example, a layer in which a layer formed of silicon oxide and a layer formed of tantalum oxide are stacked. The insulating layer 47 covers the porous oxide layer 44, the anode 45, and the solid electrolyte layer 46. The insulating layer 47 is formed with an opening that exposes the solid electrolyte layer 46. The cathode 48 is arranged on a portion of the solid electrolyte layer 46 exposed from the opening of the insulating layer 47. The cathode 48 is a layer formed of porous metal. The cathode 48 is formed of, for example, platinum.

The porous oxide layer 44, the anode 45, the solid electrolyte layer 46, the insulating layer 47, and the cathode 48 are arranged in the through-hole 42g. The porous oxide layer 34 is exposed to the cavity 31a. That is, the first element 40 has the same configuration as the second element 30.

When a voltage equal to or higher than the first voltage is applied between the cathode 48 and the anode 45, the carbon dioxide gas in the detection target gas becomes oxygen ions by receiving electrons from the cathode 48. These oxygen ions move to the anode 45 through the solid electrolyte layer 46 by a voltage between the cathode 48 and the anode 45. The oxygen ions that have moved to the anode 45 become an oxygen gas by emitting electrons to the anode 35.

A current flowing between the cathode 48 and the anode 45 due to this reaction is proportional to the concentration of the carbon dioxide gas in the detection target gas. Therefore, by detecting the current flowing between the cathode 48 and the anode 45, the first element 40 can detect the carbon dioxide concentration in the detection target gas. Since the flow rate of the detection target gas reaching the cathode 48 is not easily restricted by the first element 40, the sensitivity when the concentration of the carbon dioxide gas in the detection target gas is high is improved according to the carbon dioxide gas sensor 100B.

According to the present disclosure in some embodiments, it is possible to provide a carbon dioxide gas sensor with improved gas selectivity for a carbon dioxide gas.

While certain embodiments have been described, these embodiments have been presented by way of example only, and are not intended to limit the scope of the disclosures. Indeed, the embodiments described herein may be embodied in a variety of other forms. Furthermore, various omissions, substitutions and changes in the form of the embodiments described herein may be made without departing from the spirit of the disclosures. The accompanying claims and their equivalents are intended to cover such forms or modifications as would fall within the scope and spirit of the disclosures.

What is claimed is:

1. A carbon dioxide gas sensor comprising:
a flow path including an inlet into which a detected target gas is introduced; and
a first element and at least one second element arranged in the flow path,
wherein the first element includes a first solid electrolyte layer, a first cathode, and a first anode, the first solid electrolyte layer being interposed between the first cathode and the first anode,
wherein the at least one second element includes a second solid electrolyte layer, a second cathode, and a second anode, the second solid electrolyte layer being interposed between the second cathode and the second anode,
wherein the first solid electrolyte layer and the second solid electrolyte layer are formed of an oxygen ion conductor,
wherein the first cathode is inside the flow path, and
wherein the second cathode and the second anode are inside the flow path and outside the flow path, respectively.

2. The carbon dioxide gas sensor of claim 1, wherein a voltage, which is equal to or higher than a first voltage capable of generating oxygen ions from carbon dioxide at the first cathode, is applied between the first cathode and the first anode, and
wherein a voltage, which is equal to or higher than a second voltage capable of generating oxygen ions from water vapor, an oxygen gas, and a nitrogen oxide gas at the second cathode and lower than the first voltage, is applied between the second cathode and the second anode.

3. The carbon dioxide gas sensor of claim 1, wherein the oxygen ion conductor is yttria-stabilized zirconia.

4. The carbon dioxide gas sensor of claim 1, wherein the at least one second element is a plurality of second elements, and
wherein at least one selected from the group of the plurality of second elements is closer to the inlet than the first element in the flow path.

5. The carbon dioxide gas sensor of claim 1, wherein the first element further includes a first substrate, a first insulating layer, a first porous oxide layer, and a second insulating layer,
wherein the first substrate is formed with a first cavity that passes through the first substrate in a thickness direction,
wherein the first insulating layer is arranged on the first substrate,
wherein the first porous oxide layer is arranged on a portion of the first insulating layer on the first cavity,
wherein the first cathode is arranged on the first porous oxide layer,
wherein the first solid electrolyte layer is arranged on the first cathode,
wherein the second insulating layer is arranged on the first insulating layer so as to cover the first porous oxide layer, the first cathode, and the first solid electrolyte layer,
wherein the second insulating layer is formed with a first opening that partially exposes the first solid electrolyte layer, and
wherein the first anode is arranged on a portion of the first solid electrolyte layer exposed from the first opening.

6. The carbon dioxide gas sensor of claim 5, wherein the first porous oxide layer includes a comb tooth portion having a comb tooth shape in a plan view,
wherein the second insulating layer is removed from a side surface of the comb tooth portion, and
wherein the first cathode, the first solid electrolyte layer, and the first anode are arranged so as to overlap the comb tooth portion in a plan view.

7. The carbon dioxide gas sensor of claim 1, wherein the first element further includes a first substrate, a first insulating layer, a first porous oxide layer, and a second insulating layer,
wherein the first substrate is formed with a first cavity that passes through the first substrate in a thickness direction,
wherein the first insulating layer is arranged on the first substrate,
wherein the first anode is arranged on the first porous oxide layer,
wherein the first solid electrolyte layer is arranged on the first anode,
wherein the second insulating layer covers the first porous oxide layer, the first anode, and the first solid electrolyte layer,
wherein the second insulating layer is formed with a first opening that partially exposes the first solid electrolyte layer,
wherein the first cathode is arranged on a portion of the first solid electrolyte layer exposed from the first opening,
wherein a first through-hole configured to communicate with the first cavity is formed in a portion of the first insulating layer on the first cavity, and
wherein the first porous oxide layer, the first anode, the first solid electrolyte layer, the second insulating layer, and the first cathode are arranged in the first through-hole such that the first porous oxide layer is exposed to the first cavity.

8. The carbon dioxide gas sensor of claim 1, wherein the at least one second element further includes a second substrate, a third insulating layer, a second porous oxide layer, and a fourth insulating layer,
wherein the second substrate is formed with a second cavity that passes through the second substrate in a thickness direction,
wherein the third insulating layer is arranged on the second substrate,
wherein the second anode is arranged on the second porous oxide layer,
wherein the second solid electrolyte layer is arranged on the second anode,
wherein the fourth insulating layer covers the second porous oxide layer, the second anode, and the second solid electrolyte layer,
wherein the fourth insulating layer is formed with a second opening that partially exposes the second solid electrolyte layer,
wherein the second cathode is arranged on a portion of the second solid electrolyte layer exposed from the second opening,
wherein a second through-hole communicating with the second cavity is formed in a portion of the third insulating layer on the second cavity, and
wherein the second porous oxide layer, the second anode, the second solid electrolyte layer, the fourth insulating layer, and the second cathode are arranged in the second through-hole such that the second porous oxide layer is exposed to the second cavity.

9. A gas sensor element comprising:
a substrate;
a first insulating layer;
a porous oxide layer;
a cathode;
a solid electrolyte layer formed by an oxygen ion conductor;
a second insulating layer; and
an anode,
wherein the substrate is formed with a cavity that passes through the substrate in a thickness direction,
wherein the first insulating layer is arranged on the substrate,
wherein the porous oxide layer is arranged on a portion of the first insulating layer on the cavity,
wherein the cathode is arranged on the porous oxide layer,
wherein the solid electrolyte layer is arranged on the cathode,
wherein the second insulating layer is arranged on the first insulating layer so as to cover the porous oxide layer, the cathode, and the solid electrolyte layer,
wherein the second insulating layer is formed with an opening that partially exposes the solid electrolyte layer, and
wherein the anode is arranged on a portion of the solid electrolyte layer exposed from the opening.
